(19) United States
(12) Patent Application Publication
(10) Pub. No.: US 2021/0177818 A1
(43) Pub. Date: Jun. 17, 2021

(54) Antibacterial Formulation for Treating and Prophylactic use in Bacterial, and Fungal Urinary Bladder Infections

(71) Applicants: Ellison Van Cleef Warner, Punta Gorda, FL (US); William Randolph Warner, Punta Gorda, FL (US)

(72) Inventors: Ellison Van Cleef Warner, Punta Gorda, FL (US); William Randolph Warner, Punta Gorda, FL (US)

(21) Appl. No.: 17/170,362

(22) Filed: Feb. 8, 2021

Related U.S. Application Data
(62) Division of application No. 16/277,859, filed on Feb. 15, 2019.

(51) Int. Cl. A61K 31/4425; A61K 9/00; A61P 31/10; A61K 47/02; A61P 31/04; A61K 9/08

(52) U.S. Cl. CPC A61K 31/4425; A61K 9/0034; A61K 9/08; A61K 47/02; A61P 31/04; A61P 31/10

(57) ABSTRACT

Compositions, methods and formulation for the treatment and prophylactic management of an Antibacterial Formulation for Treating and Prophylactic use in Bacterial, and Fungal Urinary Bladder Infections include a solution comprising of Cetylpyridinium chloride and Sodium chloride in concentrations of Cetylpyridinium chloride, 0.01% to 5% and Sodium chloride, 1% to 10% at a pH of 4.5 to 8.5.

### CROSS-REFERENCE TO RELATED APPLICATIONS

This application claims the benefit of priority to U.S. provisional patent application 62/599,486, filed Dec. 17, 2017, the contents of which are herein incorporated by reference.

### FIELD OF INVENTION

The invention relates to compositions and methods of administering an instilled liquid solution into the urinary bladder, wash or lavage when indicated for Bacterial and/or Fungal Cystitis with the formulation of Cetylpyridinium chloride/hypertonic saline solutions for bacterial and fungal bladder infections. Cetylpyridinium chloride/hypertonic saline at a specific pH Ionized balance. The solution delivered using an interfaced urinary catheters or other direct bladder infusion modalities.

### BACKGROUND

### Acute and Chronic Bacterial Cystitis

Urinary tract infections (UTIs) are the most common nosocomial infections. They account for more than 7 million physician visits and over 1 million hospital admissions in the United States each year. They are the most common bacterial infection in older adults and the most frequent source of bacteremia. UTIs are second in seriousness only to respiratory infections. UTI drug resistance and chronic bacterial colonization are becoming more frequent and problematic.

Escherichia coli is the most common infecting organism in patients with uncomplicated UTIs. It causes 85% of community-acquired infections and approximately 50% of nosocomial infections.

### Klebsiella Urinary Tract Infection

The Klebsiella species form a heterogeneous group of gram negative, lactose fermenting, encapsulated, non-motile bacilli. They are important urinary tract pathogens, especially in long stay hospital patients and infection is often associated with urethral catheterization.

### Bacterial Cystitis

UTIs typically occur when bacteria outside the body enter the urinary tract through the urethra and begin to multiply. Most cases of cystitis are caused by a type of Escherichia coli (E. coli) bacteria.

### Fungal Cystitis

Among UTIs, cystitis due to Candida may result in frequency, urgency, dysuria, and suprapubic pain. Hematuria is common. In patients with poorly controlled diabetes, pneumaturia due to emphysematous cystitis has may occur. Fungus balls or bezoars may cause symptoms of urethral obstruction.

### Epidemiology

UTI is defined as significant bacteriuria in the setting of symptoms of cystitis or pyelonephritis. These infections account for a significant number of emergency department (ED) visits, and 20% of women develop at least one UTI. Escherichia coli cause the majority of uncomplicated cystitis cases.

Cystitis affects people of both sexes and all ages. It is more common among females than males because women have shorter urethras. Around 80 percent of all urinary tract infections (UTIs) are caused by bacteria from the bowel that reaches the urinary tract.

Causes of cystitis in men: The most common cause of cystitis in men is urine retention. If the bladder does not fully empty, the urine left in the bladder can become stagnant, leading to infection. This can occur because of constipation or, more commonly, an enlarged prostate.

Escherichia coli and Klebsiella infections account for the majority of Recurrent Urinary Track Infections (RUTI's) and multidrug-resistant organisms (MDROs) infections. These are bacteria. that resist treatment with more than one antibiotic. Multidrug-resistant organisms are problematic and found mainly in hospitals and long-term care facilities.

### Current Treatments

The first-choice agents for treatment of uncomplicated acute cystitis in women include nitrofurantoin monohydrate/macrocrystals, trimethoprim-sulfamethoxazole (TMP-SMX), or fosfomycin. Beta-lactam antibiotics may be used when other recommended agents cannot be used.

Due to “over prescribing” of these agents, many of Escherichia coli strains are becoming resistant to traditional drug therapy.

### SUMMARY OF THE INVENTION

The present invention provides composition and formulation for the treatment and prophylactic management of bacterial and fungal urinary bladder Infections. A solution comprising of Cetylpyridinium chloride and Sodium chloride in concentrations of Cetylpyridinium chloride, 0.01% to 5% and Sodium chloride, 1% to 10% at a pH of 4.5 to 8.5.

Additional features, advantages and embodiments of the invention may be set forth or apparent for consideration of the following description and claims. Moreover, it is to be understood that both the foregoing summary of the invention and the following description are exemplary and intended to prove further explanation without limiting the scope of the invention claimed.

### DETAILED DESCRIPTION

Embodiments of the invention relate to a compound, solution, formulation of Cetylpyridinium chloride and Sodium chloride.

The composition of active agents of the invention include Cetylpyridinium chloride, 0.01% to 5.0% and Sodium chloride, 1% to 10% (hypertonic solution) with pH ionization at a pH of 4.5 to 8.5.

### Cetylpyridinium Chloride (CPC)

Cetylpyridinium chloride is a cationic quaternary ammonium compound. CPC is used as a an antiseptic, antibacterial agent in some types of mouthwashes, toothpastes, lozenges, throat sprays, anti-sore throat sprays, breath sprays, antibacterial/antifungal nasal sprays and as an antibacterial, antimicrobial preservative in pharmaceuticals. It is an antiseptic that kills bacteria and other microorganisms.

Cetylpyridinium chloride has the molecular formula C₂₁H₃₈NCl and at its pure form is in a solid at room temperature. It has a melting point of 77 ° C. when anhydrous or 80-83° C. in its monohydrate form. It is insoluble in acetone, acetic acid, or ethanol. It has a pyridine-like odor.

Cetylpyridinium chloride is a quaternary nitrogenous compound, 1-hexa-decyl pyridinium chloride, with antimicrobial activity. The compound is classified as a cationic surface-active that renders molecules lypophilic, an attribute necessary for the antimicrobial activity.

### Mechanism of Action

Cetylpyridinium chloride (CPC) is a surface-active cationic antiseptic, with antimicrobial activity against bacteria, fungi, and viruses. Cetylpyridinium chloride binds to the cell membrane. This binding disrupts osmotic integrity of the cellular membrane, resulting in leakage of intracellular potassium, magnesium, sugars, and metabolites and then cellular death.

### Sodium chloride

Saline (also saline solution) is a general term referring to a sterile solution of sodium chloride in water. It is used for intravenous infusion, rinsing contact lenses, and irrigation and inhaled forms.

As used herein, the term “saline” refers to salt, usually Sodium chloride, but can include salt of potassium, magnesium or calcium. Physiological saline contains 0.9% of Sodium chloride in water and is isotonic (i.e. having same osmotic pressure as blood serum).

As used herein, the term “hypertonic” refers to a solution with a solute concentration that is higher than that inside cells present in that solution, and therefore causes water to diffuse out of the cells. The term “hypertonic” is a relational term expressing the greater relative solute concentration of one solution compared with another (i.e., the latter is “hypertonic” to the former). A hypertonic solution has a lower water potential than a solution that is hypotonic to it and has a correspondingly greater osmotic pressure.

As used herein, the term “osmotic activity” refers to the net diffusion of water across a selectivity permeable membrane that is permeable in both directions to water, but varying permeable to solutes, wherein the water diffuses from one solution into another of lower water potential.

### Mechanism of Action

Hypertonic saline solutions in concentrations greater than 1% have shown to be bactericidal and bacteriostatic. In general, if an antibacterial agent is bacteriostatic, it means that the agent essentially stops bacterial cell growth (but does not kill the bacteria); if the agent is bactericidal, it means that the agent kills the bacterial cell (and may stop growth before killing the bacteria).

### Combination Formulation

In embodiments of the present invention, the combination of Cetylpyridinium chloride and hypertonic Sodium chloride have synergistic effects.

The hypertonic Sodium chloride's osmotic property creates mobilization or motility of bacterial and fungal pathogens that harbor in the urinary bladder. The solute concentration causes water to diffuse out of the cells.

The mobilization or motility of bacterial and fungal pathogens creates greater surface exposure of the bacterial and fungal pathogens allowing the

Cetylpyridinium chloride to bind to the cell membranes disrupting the osmotic integrity of the cellular membrane, causing leakage of metabolites and then cellular death.

### Synergistic Effect

It is not uncommon for the effect of two chemicals on an organism to be greater than the effect of each chemical individually, or the sum of the individual effects. The presence of one chemical enhances the effects of the second is a synergistic effect. A significant embodiment of the invention relates to the synergistic antimicrobial activity of the combination of Cetylpyridinium chloride and Sodium chloride and with modified pH ionization.

Alone, Cetylpyridinium chloride is a surface-active cationic antiseptic, with antimicrobial activity against bacteria, fungi, and viruses. Cetylpyridinium chloride binds to the cell membrane. This binding disrupts osmotic integrity of the cellular membrane, resulting in leakage of intracellular potassium, magnesium, sugars, and metabolites and then cellular death.

Alone, hypertonic Sodium chloride solutions in concentrations greater than 1% (w/w) have shown to be bactericidal and bacteriostatic.

Without being held to any particular theory, the combined synergistic effects of the Cetylpyridinium chloride and Sodium chloride according to embodiments of the present invention have been found to be three-fold. The hypertonic Sodium chloride osmotically lowers the viscosity of the bladder fluids allowing greater surface contact of the Cetylpyridinium chloride to the microorganisms where the cationic effect of the Cetylpyridinium chloride disrupts osmotic integrity of the cellular membranes. The bactericidal properties of both Cetylpyridinium chloride and Sodium chloride are synergistically enhanced with the osmotically of the hypertonic of the Sodium chloride to draw-out cellular metabolites of the microorganisms' cell membranes that have been disrupted or breached by the cationic effects of the Cetylpyridinium chloride.

This synergistic effect of the invention exposes more pathogens to the combination-agent and provides a greater and quicker “Kill” effect of the cells than if used as a single or separate agent alone.

### pH Ionization

Another embodiment of the invention relates to the synergistic antimicrobial enhancement of the connexin hemichannel.

pH ionization also known as a connexin hemichannel, is an assembly of six proteins called connexins that form the pore for a gap junction between the cytoplasm of two adjacent cells. This channel allows for bidirectional flow of ions and signaling molecules that enhances the synergic properties of the biofilm cellular disruption of the formulation.

The pH of urine can range from an extremely unhealthy low of 4.5 to a high of 8.5.

In a pH balanced body, urine is slightly acid in the morning (after fasting), with a pH range of 6.5-7.0, generally becoming more alkaline, pH=7.5−8.0, by evening as the body digests food and releases electrolytes.

The average person consumes an acidifying diet and on average has a urine pH of 5.5, whereas it is generally preferable to be at 7.0-7.5, an alkaline environment that enhances connexin molecule function when the urine pH is below 7.0.

In a recent In-vitro study on five E. coli human recurrent urinary tract infection isolates using two different batches of the solution was observed, the connexin hemi channel allowed the complete killing of these bacterial isolates with 20% and 30% of solutions according to embodiments of the present invention within 15 minutes of exposure at a pH of 5.5 to 8.5. Regardless of the of the solution's pH or the pH of the test samples, the connexin hemichannel helped allowed a bidirectional, uniform kill rate of the bacteria tested with a pH of 5.5 to 8.5, was noted. The above study was performed with a solution of 0.01% (w/w) CPC in 1.25% aqueous NaCl.

### Surfactants

Surfactants are commonly used in liquid formulations. They include alkylbenzene sulfonates (detergents), (fatty acid) soaps, lauryl sulfate (foaming agent), di-alkyl sulfosuccinate (wetting agent), lignosulfonates (dispersants) etc. Anionic surfactants account for about 50% of the world production.

The use of USP surfactants may be incorporated into the formulation for stability and/or reduce foaming.

### Antibacterial Antifungal Spectrum

The invention is a compound, solution, formulation of Cetylpyridinium chloride and Sodium chloride has shown in-vitro and/or vivo to be effective against gram positive, gram negative bacteria and gram neutral such as Staphylococcus aureus, Pseudomonas aeruginosa, Streptococcus pyogenes, Haemophilus influenzae, Listeria monocytogenes, Moraxella cattarrhalis, Salmonella typhimurium, Mycobacterium tuberculosis and E. coli, Klebsiella and the fungi Pneumocystis jirovecii, Alternaria alternata, Aspergillus niger, Penicillium funiculosum, Fusarium solani, and Candida albicans.

### Concomitant Use

As found in the section “Current Treatments”, most bacterial and fungal bladder infections are treated with oral systemic (tablets, liquids, syrups) or injectable anti-infective medications. The oral systemic tablets, liquids, syrups must be carried to the pathogen via the blood stream and requiring cellular uptake by the infective cells to be effective. Injectable anti-infective medications must also be carried to the pathogen via the blood stream to the infected cells to be effective.

As in the invention, the intraurethral bladder installation, interfaces with an intraurethral catheter, (IV Bag or Lg volume syringe) wash or lavage the pharmacological properties of the combination of Cetylpyridinium chloride and hypertonic Sodium Chloride have direct cellular contact without having to be ingested, IM/IV injected or metabolize. The direct infusion via an intraurethral catheter modality delivers the active agents directly to the bladder where the bacterial and fungal pathogens colonize. Thus, the invention having a separate delivery pathway and antibacterial, antifungal activity than that of systemically delivered agents, would allow for concomitant use.

### E. coli In-Vitro Clinical Data

### Materials and Methods

The efficacy of compositions of the present invention against uropathogenic E. coli was assessed using minimum inhibitory concentration (MIC) determination assays and time-kill assays in standard laboratory medium (Luria-Bertani medium) and media with a range of different pH representative of urine pH variability (pH 5.5, 6.5, 7.5 and 8.5). Colony forming units (CFU)/mL and optical density (turbidity) were used to assess bacterial growth. Five different uropathogenic E. coli strains obtained from RUTI patients, referred to as RUTI-12, PNK 16, PNK 29, PNK 30, PNK 45, were assessed.

### Results

A similar effectiveness of compositions of the present invention on five E. coli human recurrent urinary tract infection isolates using two different batches of the solution was observed. The complete killing of these bacterial isolates with 20% and 30% KG 7656 solutions within 15 minutes of exposure, regardless of the pH (5.5 to 8.5), was also noted.

### CONCLUSION

When assessed for MIC and time-kill rate, compositions of the present invention exhibited a potent effect on human uropathogenic E. coli strains, justifying consideration of its selective testing in vivo.

### Definitions

The terms “active agent,” “drug” and “pharmacologically active agent” are used interchangeably herein to refer to a chemical material or compound which, when administered to an organism (human or animal) induces a desired pharmacologic effect. Included are derivatives and analogs of those compounds or classes of compounds specifically mentioned that also induce the desired pharmacologic effect.

By “pharmaceutically acceptable carrier” is meant a material or materials that are suitable from drug administration and not biologically or otherwise undesirable, i.e., that may be administered to an individual along with an active agent without causing any undesirable biological effects or interacting in a deleterious manner with any of the other components of the pharmaceutical formulation in which it is contained.

Similarly, a “pharmacologically acceptable” salt, ester or other derivative of an active agent as provided herein is a salt, ester or other derivative that is not biologically or otherwise undesirable.

By the terms “effective amount” or “therapeutically effective amount” of an agent as provided herein are meant to be nontoxic but in a sufficient amount of the agent to provide the desired therapeutic effect. The exact amount required will vary from subject to subject, depending on the age, weight, general condition and bio-burden of the subject and the severity of the condition being treated, the judgment of the clinician should determine the effective amount and duration of treatment on an individual case. Thus, it is not possible to specify an exact “effective amount or specific volume and time of treatment”.

By the term “synergic effect” meaning that when the chemical, or compound that work together, they accomplish more than they could alone. Synergetic is often used to describe the effect of drugs working together—where one chemical, or compound increases the other's effectiveness.

The terms “treating” and “treatment” and ‘Prophylactic” as used herein refer to reduction in severity and/or frequency of symptoms, elimination of symptoms and/or underlying cause, prevention of the occurrence of symptoms and/or their underlying cause, and improvement or remediation of damage. Thus, for example, the present method of “treating” individuals for a treatment and prophylactic management of an Antibacterial Formulation for Treating and Prophylactic use in Bacterial, and Fungal Urinary Bladder Infections. The term “treating” is used herein, encompasses treatment to clinically symptomatic individual.

According to another embodiment, the Cetylpyridinium chloride/hypertonic saline solution of the invention may have a pH in the range of about pH 4.5 to about pH 8.5,

The pH may be adjusted by the addition of a solution containing an acidic salt or an acid (e.g., hydrochloric or sulfuric acid); or of the basic salt or a base (e.g., sodium hydroxide). In case of insufficient stability of the formulation, aqueous buffered systems (citrate, acetate, phosphate) may be added to keep the pH with a physiologically acceptable range. A variety of buffers known in the art may be used in the formulation of the invention, such as various salts of organic or inorganic acids, bases, or amino acids, and including various forms of citrate, phosphate, tartrate, succinate, adipate, maleate, lactate, acetate, bicarbonate, or carbonate ions.

What is claimed is:

1. A method for treating or preventing a urinary bladder infection, comprising administering to a patient in need thereof a therapeutically effective amount of a composition comprising cetylpyridinium chloride and a hypertonic sodium chloride solution.

2. The method of claim 1, further comprising delivering the composition to a urinary bladder of the patient via a urinary catheter.

3. The method of claim 1, further comprising washing or lavaging the composition in a urinary bladder of the patient.

4. The method of claim 1, wherein the urinary bladder infection is at least one or a bacterial infection and a fungal infection.

5. The method of claim 1, wherein the concentration of the cetylpyridinium chloride is in the range of about 0.01% to about 5%.

6. The method of claim 1, wherein the concentration of Sodium chloride is in the range of about 1% to about 10%.

7. The method of claim 1, wherein the cetylpyridinium chloride acts as a surface active cationic antiseptic, with antimicrobial activity against bacteria, fungi, and viruses.

8. The method of claim 1, wherein the hypertonic sodium chloride has an osmotic property that creates mobilization or motility of bacterial and fungal pathogens that colonize in the bladder.
